(19) United States
(12) Patent Application Publication
(10) Pub. No.: US 2017/0156429 A1
(43) Pub. Date: Jun. 8, 2017

(54) BALDING CONCEALEMENT METHOD AND APPARATUS

(71) Applicant: Salvatore Ronnie Passariello, Lynnfield, MA (US)

(72) Inventor: Salvatore Ronnie Passariello, Lynnfield, MA (US)

(21) Appl. No.: 14/957,161

(22) Filed: Dec. 2, 2015

(51) Int. Cl. A41G 5/00; A61Q 5/00; A61K 8/98

(52) U.S. Cl. CPC A41G 5/0013; A61K 8/985; A61Q 5/00; A61K 2800/87

(57) ABSTRACT

A hair concealment method and apparatus is provided. The hair concealment apparatus includes a container, a cap, and a plurality of animal hairs disposed within the container. The static electricity is removed from the plurality of animal hairs and applied to the scalp of the user.

### BACKGROUND OF THE INVENTION

The present invention relates to a balding remedy and, more particularly, to human hairs within a portable container.

Hair loss, also known as alopecia or baldness, refers to a loss of hair from the head. Baldness can refer to general hair loss or male pattern haft loss. Male-pattern hair loss is hair loss that occurs due to an underlying susceptibility of haft follicles to androgenic miniaturization. It is the most common cause of haft loss and will affect up to 70% of men and 40% of women at some point in theft lifetimes.

As can be seen, there is a need for a balding concealment method and apparatus.

### SUMMARY OF THE INVENTION

In one aspect of the present invention, a method of concealing baldness comprises: removing a static electricity from a plurality of animal hairs; and placing the plurality of animal hairs on a bald spot of a head of a user.

In another aspect of the present invention, a hair concealment apparatus comprises: a container comprising an entrance leading into the container; a cap comprising a plurality of apertures forming a screen, wherein the cap is secured to the entrance; and a plurality of animal hairs within the container, the plurality of animal hairs having had a static electricity removed, wherein the plurality of apertures are sized to permit the plurality of animal hairs to pass through.

These and other features, aspects and advantages of the present invention will become better understood with reference to the following drawings, description and claims.

### BRIEF DESCRIPTION OF THE DRAWINGS

FIG. 1 is a profile view demonstrating an exemplary affected region of a user's head;

FIG. 2 is a profile view demonstrating the application of an embodiment of the present invention to the exemplary affected region;

FIG. 3 is detail section view of an exemplary affected region; and

FIG. 4 is a detail section view demonstrating application of an embodiment of the present invention to the exemplary affected region.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes human hair fibers in a bottle to be used for the purpose of concealing thinning hair. The human hair fiber of the present invention when applied conceals thin or bald spots on the head. The present invention may include 100% human hair fibers and because of this, the product looks more natural, covers better, and stays in place longer than other balding remedies.

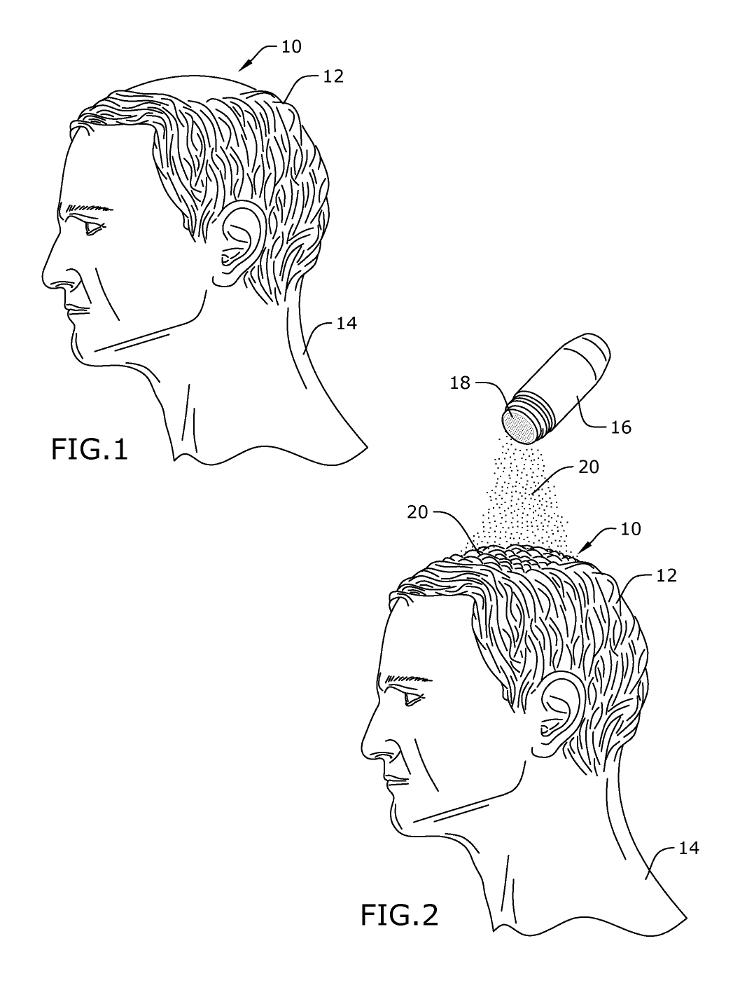

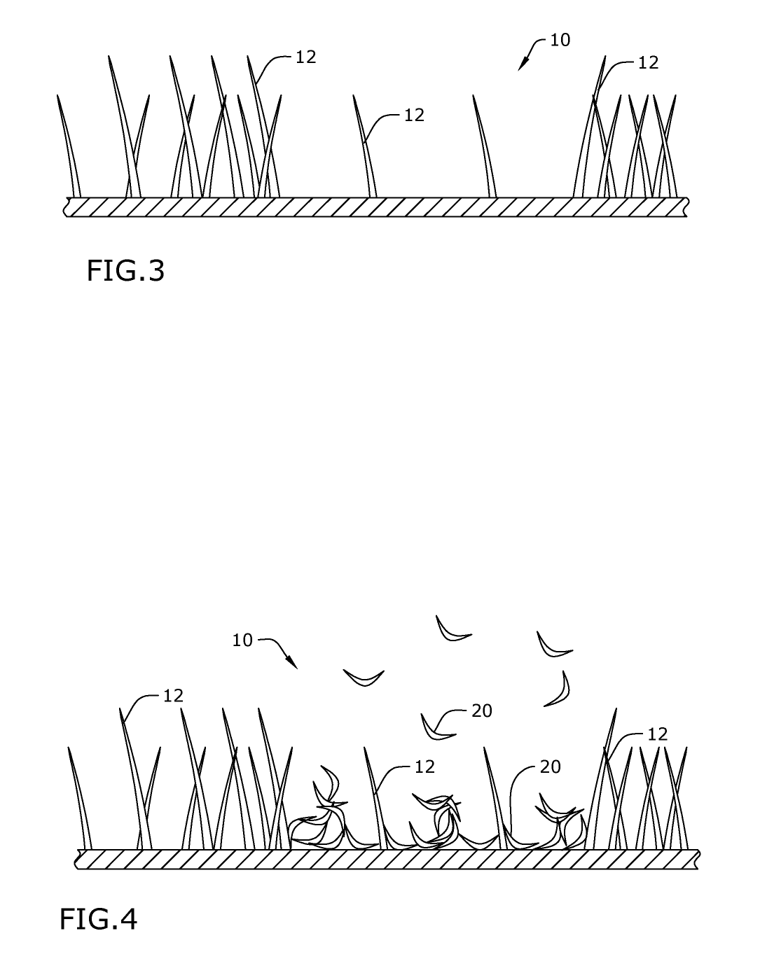

Referring to FIGS. 1 through 4, the present invention includes a hair concealment apparatus. The hair concealment apparatus includes a container 16, a cap 18, and a plurality of animal hairs 20 disposed within the container 16.

The container 16 of the present invention includes an entrance that leads into an inner housing. The cap 18 includes a plurality of apertures forming a screen. The cap 18 is releasably securable to the container 16 and covers the entrance of the container 16. The cap 18 may be releasably securable to the container via mating threads, snaps, or other connectors. The plurality of apertures are sized to permit the plurality of animal hairs 20 to pass through. The apertures of the cap 18 may allow the hair fibers 20 to dispense at a controlled rate. The cap 18 seals the container 16 to prevent leakage when not in use.

The plurality of animal hairs 20 may include a plurality of human hairs. The static electricity of the plurality of animal hairs 20 is removed from the plurality of animal hairs 20 such that the plurality of animals hairs 20 are attracted to the existing human hair 12, and thereby retain to the head of the user 14. Further, the present invention may be used in conjunction with a holding spray/hair spray to allow the product to have a more firm hold. The present invention may further work in conjunction with a tool to help create a straight line with the fiber.

To use the present invention, the following steps may be performed. The static is first removed from the plurality of animal hairs 20 and placed within the container 16. The cap 18 is attached to the container 16 to cover the entrance of the container. A user 14 may then shake the container 16 over an alopecia patch 10 such that the plurality of animal hairs 20 falls through the apertures formed through the cap 18. The plurality of hair fibers 20 then secure to the existing hair 12 of the user 14 and conceal the alopecia patch 10 of the user 14.

The present invention may be made from human hair fibers, the same hair that is currently being used in wigs. The hair may be washed, dried, died a specific color and ground into the appropriate size. The hair is then placed into the container where it is sealed with a tamper proof seal and then the cap/cover is applied. Place the present invention above the thin hair spot a user would like to conceal, turn the container upside down and shake so the fibers come out as needed. Thousands of microscopic hair fibers may be released and blended with the user's natural hair.

It should be understood, of course, that the foregoing relates to exemplary embodiments of the invention and that modifications may be made without departing from the spirit and scope of the invention as set forth in the following claims.

What is claimed is:

1. A method of concealing baldness comprising:
removing a static electricity from a plurality of animal hairs; and
placing the plurality of animal hairs on a bald spot of a head of a user.

2. The method of claim 1, wherein the plurality of animal hairs is a plurality of human hair.

3. The method of claim 1, further comprising the steps of:
containing the plurality of animal hairs within a container after the step of removing the static electricity from the plurality of animal hairs.

4. The method of claim 3, wherein the container further comprises a cap releasably retained to an entrance of the container, wherein the cap comprises a plurality of apertures forming a screen.

5. The method of claim 4, further comprising the step of:
shaking the container over the bald spot such that the cap is facing the bald spot and thereby delivering the plurality of animal hair.

6. A hair concealment apparatus comprising:
a container comprising an entrance leading into the container;
a cap comprising a plurality of apertures forming a screen, wherein the cap is secured to the entrance; and
a plurality of animal hairs within the container, the plurality of animal hairs having had a static electricity removed, wherein
the plurality of apertures are sized to permit the plurality of animal hairs to pass through.

7. The hair concealment apparatus of claim 6, wherein the plurality of animal hair is a plurality of human hair.

8. The hair concealment apparatus of claim 6, wherein the cap is releasably secured to the entrance of the container.
